(19) United States
(12) Patent Application Publication
(10) Pub. No.: US 2008/0208064 A1
(43) Pub. Date: Aug. 28, 2008

(54) Method and apparatus for measuring blood pressure

(76) Inventor: Shao-Hung Lee, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/710,936

(22) Filed: Feb. 27, 2007

(51) Int. Cl. A61B 5/021

(52) U.S. Cl. 600/485

(57) ABSTRACT

A method and an apparatus for measuring blood pressure are disclosed. The method is applied to a blood pressure measurement apparatus and comprises the steps of providing a sensing unit for sensing a user's pulse, counting the pulse in a time interval by a counting unit based on the sensing unit to calculate a pulse rate, determining the condition of the pulse rate by a determination mechanism to generate an operation signal, controlling the blood pressure measurement apparatus based on the operation signal to determine whether or not the pulse rate is steady to ensure the accuracy of measuring the blood pressure. Therefore, the invention can improve the inaccuracy of the blood pressure measurement caused by human errors.

### BACKGROUND OF THE INVENTION

1. Field of the Invention

The present invention relates to a method and an apparatus for measuring blood pressure, and more particularly to a technical area of determining whether or not a blood pressure is measured in a stable condition.

2. Description of the Related Art

Blood pressure is the pressure exerted by the blood against the walls of blood vessels, and the blood coming from the heart requires a pushing force to circulate around a human body, and the heart keeps on contracting and expanding to push the blood forward. In general, a blood pressure can provide the following physiological information: 1. Systolic pressure, which is an upper reading of blood pressure measured while the heart is contracting to pump blood at the blood vessel. 2. Diastolic pressure, which is a lower reading of blood pressure measured while the heart is not contracting. 3. Pulse which is the number of contractions of the heart per minute. Users can know about their health conditions from these important indexes by examining whether or not the measured readings exceed a normal range.

The conventional electronic sphygmomanometers generally measure the systolic pressure, the diastolic pressure and the pulse, but most of the present electronic sphygmomanometer provides independent readings, and users have to determine whether or not these readings exceed the normal range on their own in order to know their health conditions. Furthermore, users have to take a rest for several minutes first before taking a blood pressure measurement to ensure an accurate measurement. However, various different physiological parameters are affected by the physical exercises or the fluctuation of psychological status of a user, and thus it is an important research subject for related manufacturers to find a way of measuring the physiological parameters accurately.

In view of shortcomings of the prior art, the inventor of the present invention based on years of experience in the medical measuring apparatus related industry to conduct extensive researches and experiments, and finally developed a method and an apparatus for measuring blood pressure in accordance with the present invention to overcome the aforementioned shortcomings.

### SUMMARY OF THE INVENTION

Therefore, it is a primary object of the present invention to provide a method and an apparatus for measuring blood pressure, and specifically a technical area of determining whether or not a blood pressure is measured in a stable condition.

To achieve the foregoing object, the present invention provides a method for measuring blood pressure, and the method is applied to a blood pressure measurement apparatus and comprises the steps of: providing a sensing unit for sensing a user's pulse; counting the pulse in a time interval by a counting unit based on the sensing unit to calculate a pulse rate; determining the condition of the pulse rate by a determination mechanism to generate an operation signal; and controlling the blood pressure measurement apparatus based on the operation signal.

The present invention provides a blood pressure measurement apparatus in addition to a method for measuring blood pressure, and the blood pressure measurement apparatus comprises a blood pressure measurement body, a sensing unit, a counting unit and a processing unit, wherein the blood pressure measurement body is provided for measuring a user's blood pressure, and the sensing unit is provided for sensing a user's pulse, and the counting unit is provided for counting the pulse in a time interval based on the sensing unit to calculate a pulse rate, and the processing unit is provided for executing a determination mechanism to determine the condition of the pulse rate to generate an operation signal for controlling the measurement conducted by the blood pressure measurement body.

Since the method and apparatus of measuring blood pressure in accordance with the present invention determine whether or not the present pulse rate of a user is stable to ensure the accuracy of a blood pressure measurement, therefore the invention can improve the inaccuracy of the blood pressure measurement caused by human errors.

To make it easier for our examiner to understand the technical characteristics and effects of the present invention, we use preferred embodiments together with the attached drawings for the detailed description of the present invention.

### BRIEF DESCRIPTION OF THE DRAWINGS

FIG. 1 is a flow chart of a method for measuring blood pressure in accordance with the present invention;

FIG. 2 is a flow chart of a method for measuring blood pressure in accordance with a preferred embodiment of the present invention;

FIG. 3 is a schematic block diagram of an apparatus for measuring blood pressure in accordance with the present invention; and

FIG. 4 is a flow chart of another method for measuring blood pressure in accordance with the present invention.

### DETAILED DESCRIPTION OF THE PREFERRED EMBODIMENTS

In describing a preferred embodiment of the method and apparatus for measuring blood pressure in accordance with the invention illustrated in the drawings, specific terminologies and numerals will be resorted to for the sake of clarity. However, the invention is not intended to be limited to the specific terms and numerals so selected, and it is to be understood that each specific term includes all technical equivalents which operate in a similar manner to accomplish a similar purpose.

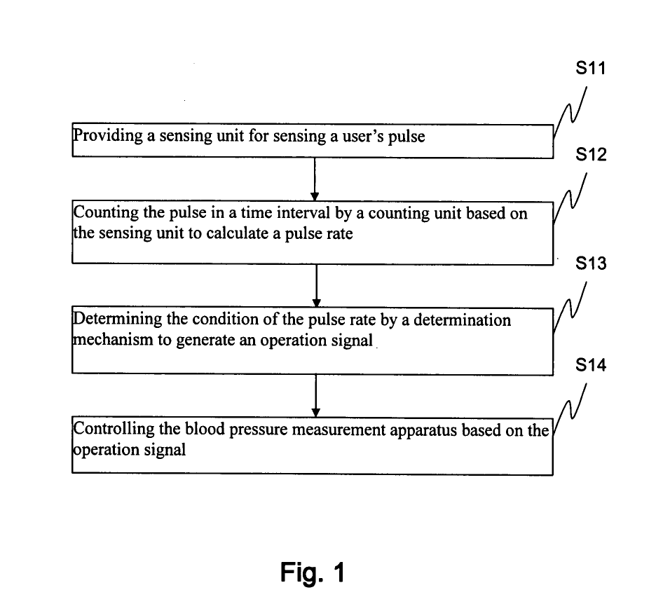

Referring to FIG. 1 for a flow chart of a method for measuring blood pressure in accordance with the present invention, the method is applied to a blood pressure measurement apparatus, and comprises the steps of:

Step S11: providing a sensing unit for sensing a user's pulse;

Step S12: counting the pulse in a time interval by a counting unit based on the sensing unit to calculate a pulse rate;

Step S13: determining the condition of the pulse rate by a determination mechanism to generate an operation signal; and

Step S14: controlling the blood pressure measurement apparatus based on the operation signal.

The determination mechanism generally includes a program, for determining whether the pulse rate is stable or unstable based on a change interval corresponding to a change of the pulse rate. The change interval is measured in a unit of a pulse number or a percentage. If the pulse rate is stable, the operation signal includes an operation of measuring a blood pressure measurement or reminding a user to take a blood pressure measurement. If the pulse rate is unstable, the operation signal includes an operation of prohibiting a blood pressure measurement or reminding a user about the unstable pulse. Further, the method for measuring blood pressure further provides a control unit for compulsorily issuing an operation signal for performing a blood pressure measurement.

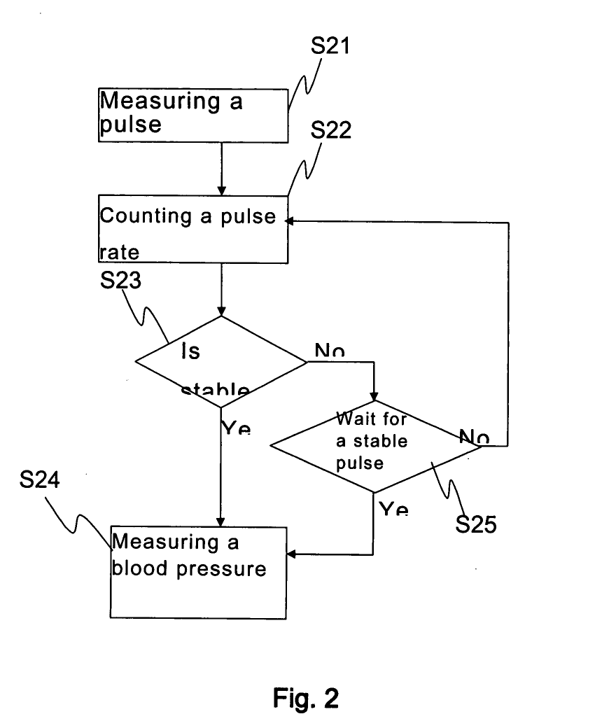

Referring to FIG. 2 for a flow chart of measuring blood pressure in accordance with a preferred embodiment of the present invention, the method for measuring blood pressure comprises the steps of:

Step S21: measuring a user's pulse;

Step S22: counting a user's pulse rate;

Step S23: determining whether or not the pulse rate is stable; if yes, then go to Step S24, or else go to Step S25;

Step S24: performing a blood pressure measurement; and

Step S25: selecting whether or not to wait for a stable pulse; if yes, return to Step S22, or else go to Step S24.

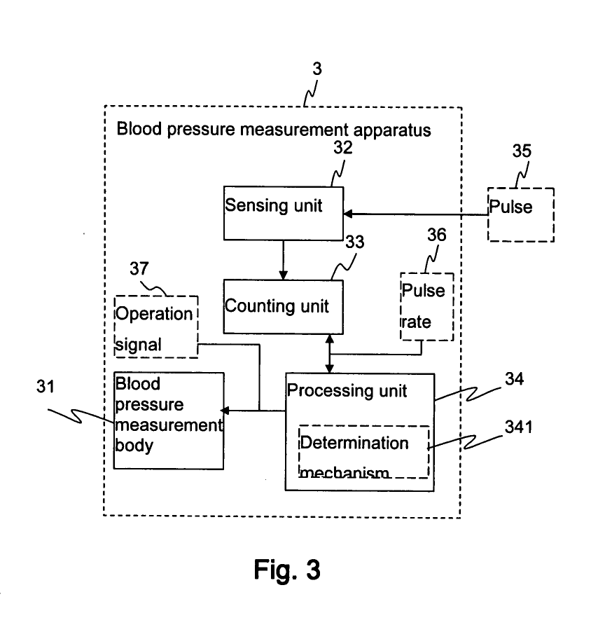

Referring to FIG. 3 for a schematic block diagram of a blood pressure measurement apparatus in accordance with the present invention, the blood pressure measurement apparatus 3 comprises a blood pressure measurement body 31, a sensing unit 32, a counting unit 33 and a processing unit 34, wherein the blood pressure measurement body 31 is provided for measuring a user's blood pressure, and the sensing unit 32 is provided for sensing a user's pulse 35, and the counting unit 33 is provided for counting the pulse in a time interval based on the sensing unit 32 to calculate a pulse rate 36, and the processing unit 34 is provided for executing a determination mechanism 341 to determine the condition of the pulse rate 36 to generate an operation signal 37 for controlling a blood pressure measurement body 31 to perform a measurement or generate a reliable blood pressure information.

The determination mechanism 341 generally includes a program for determining whether the pulse rate 36 is stable or unstable by a change interval corresponding to a change of the pulse rate 36, and the change interval is measured in a unit of a pulse number or a percentage. If the pulse rate 36 is stable, the operation signal 37 includes an operation of measuring a blood pressure measurement or generating reliable information. If the pulse rate 36 is unstable, the operation signal 37 includes an operation of prohibiting a blood pressure measurement or reminding a user about the unstable pulse or generating unreliable information. Further, the method for measuring blood pressure further provides a control unit for compulsorily issuing an operation signal 37 for performing a blood pressure measurement.

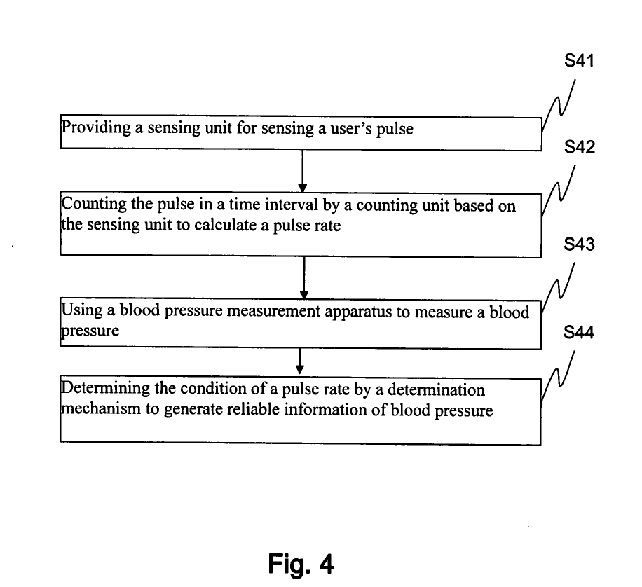

Referring to FIG. 4 for a schematic block diagram of a blood pressure measurement apparatus in accordance with the present invention, the method for measuring blood pressure is applied to a blood pressure measurement apparatus, and comprises the steps of:

Step S41: providing a sensing unit for sensing a user's pulse;

Step S42: counting the pulse in a time interval by a counting unit based on the sensing unit to calculate a pulse rate;

Step S43: using a blood pressure measurement apparatus to measure a blood pressure; and

Step S44: determining the condition of a pulse rate by a determination mechanism to generate reliable information of blood pressure.

The determination mechanism generally includes a program for determining whether the pulse rate is stable or unstable by a change interval corresponding to a change of the pulse rate, and the change interval is measured in a unit of a pulse number or a percentage. If the pulse rate is stable, reliable information will be generated. If the pulse rate is unstable, unreliable information will be generated.

While the invention has been described by means of specific embodiments, numerous modifications and variations could be made thereto by those skilled in the art without departing from the scope and spirit of the invention set forth in the claims.

What is claimed is:

1. A method for measuring blood pressure measurement, applied to a blood pressure measurement apparatus, and said method comprising the steps of:
providing a sensing unit for sensing a user's pulse;
counting the pulse in a time interval by a counting unit based on said sensing unit to calculate a pulse rate;
determining the condition of said pulse rate by a determination mechanism to generate an operation signal; and
controlling said blood pressure measurement apparatus based on said operation signal.

2. The method for measuring blood pressure of claim 1, wherein said determination mechanism includes a program.

3. The method for measuring blood pressure of claim 1, wherein said determination mechanism determines whether said pulse rate is stable or unstable based on a change interval corresponding to a change of said pulse rate.

4. The method for measuring blood pressure of claim 3, wherein said change interval is measured in a unit of a pulse number or a percentage.

5. The method for measuring blood pressure of claim 3, wherein said operation signal controls an operation of measuring blood pressure or reminding a user to carry out a blood pressure measurement if said pulse rate is stable.

6. The method for measuring blood pressure of claim 3, wherein said operation signal controls an operation of prohibiting a blood pressure measurement or reminding a user about an unstable pulse if said pulse rate is unstable.

7. The method for measuring blood pressure of claim 6, wherein the method further comprises the step of providing a control unit for compulsorily issuing said operation signal to carry out a blood pressure measurement.

8. A method for measuring blood pressure, applicable for a blood pressure measurement apparatus, and said method comprising the steps of:
providing a sensing unit for sensing a user's pulse;
counting the pulse in a time interval by a counting unit based on said sensing unit to calculate a pulse rate;
using said blood pressure measurement apparatus to measure a blood pressure; and
determining the condition of said pulse rate by a determination mechanism to generate reliable information of said blood pressure.

9. The method for measuring blood pressure of claim 8, wherein said determination mechanism includes a program.

10. The method for measuring blood pressure of claim 8, wherein said determination mechanism determines whether said pulse rate is stable or unstable based on a change interval corresponding to a change of said pulse rate.

11. The method for measuring blood pressure of claim 10, wherein said change interval is measured in a unit of a pulse number or a percentage.

12. The method for measuring blood pressure of claim 10, wherein the method further comprises a step of generating said reliable information, if said pulse rate is stable.

13. The method for measuring blood pressure of claim 10, wherein the method further comprises a step of generating unreliable information if said pulse rate is unstable.

14. A blood pressure measurement apparatus, comprising:
a blood pressure measurement body for measuring a user's a blood pressure;
a sensing unit for sensing a user's pulse;
a counting unit for counting the pulse in a time interval based on said sensing unit to calculate a pulse rate; and
a processing unit for executing a determination mechanism to determine the condition of said pulse rate to generate an operation signal for controlling said blood pressure measurement body to carry out a measurement or generate reliable information of said blood pressure.

15. The blood pressure measurement apparatus of claim 14, wherein said determination mechanism includes a program.

16. The blood pressure measurement apparatus of claim 14, wherein said determination mechanism determines whether said pulse rate is stable or unstable according to a change interval corresponding to a change of said pulse rate.

17. The blood pressure measurement apparatus of claim 16, wherein said change interval is measured in a unit of a pulse number or a percentage.

18. The blood pressure measurement apparatus of claim 16, wherein said operation signal includes an operation of performing a blood pressure measurement, or reminding a user to perform a blood pressure measurement, or generating said reliable information if said pulse rate is stable.

19. The blood pressure measurement apparatus of claim 16, wherein said operation signal includes an operation of prohibiting a blood pressure measurement, or reminding a user about an unstable pulse, or generating unreliable information if said pulse rate is unstable.

20. The blood pressure measurement apparatus of claim 19, wherein the apparatus further comprises a control unit for compulsorily issuing said operation signal to carry out said blood pressure measurement.
